SEQUENCE LISTING (8 sequences)

SEQ ID NO: 1 - DNA, 60 bp, Artificial
  Synthetic Probe
tcggttaaggaagctgtccaggcccttgagaagttctttggggtctatgggacccgaacc

SEQ ID NO: 2 - DNA, 60 bp, Artificial
  Synthetic Probe
gccgtatctgaggaacagcagcctgcactcaagggcaaaaagggaaaggaagagaagtca

SEQ ID NO: 3 - DNA, 60 bp, Artificial
  Synthetic Probe
ccgaaacgaaagaagcagaatcatcaccagccaccgacacagcagcagcccccgctgccc

SEQ ID NO: 4 - DNA, 60 bp, Artificial
  Synthetic Probe
ctcccgtttgtcctgcaactgcttcttcttctgcttcacgctgaatggcttcttcctcgg

SEQ ID NO: 5 - DNA, 58 bp, Artificial
  Synthetic Oligonucleotide
aaatgatacggcgaccaccgagatctacactctttccctacacgacgctcttccgatc

SEQ ID NO: 6 - DNA, 21 bp, Artificial
  Synthetic Oligonucleotide
gatcggaagagcgtcgtgtag

SEQ ID NO: 7 - DNA, 20 bp, Artificial
  Synthetic Oligonucleotide
aatgatacggcgaccaccga

SEQ ID NO: 8 - DNA, 21 bp, Artificial
  Common Probe 5′ Sequence
caagcagaagacggcatacga

(19) United States
(12) Patent Application Publication
(10) Pub. No.: US 2007/0141604 A1
(43) Pub. Date: Jun. 21, 2007

(54) Method of target enrichment

(76) Inventors: Niall Anthony Gormley, Waldon (GB); John Stephen West, Waldon (GB)

Correspondence Address: KLAUBER & JACKSON, 411 HACKENSACK AVENUE, HACKENSACK, NJ, 07601, US

(21) Appl. No.: 11/599,605

(22) Filed: Nov. 14, 2006

Related U.S. Application Data
(60) Provisional application No. 60/837,108, filed on Aug. 10, 2006.
(60) Provisional application No. 60/736,785, filed on Nov. 15, 2005.

(51) Int. Cl. C12Q 1/68; C12P 19/34

(52) U.S. Cl. 435/6; 435/91.2

(57) ABSTRACT

The present invention is directed to a method for reducing the complexity of a nucleic acid sample in a reproducible manner by enriching for specific nucleic acid target sequences in the population of nucleic acids. More specifically, the invention relates to a method for enriching specific target sequences in a population using libraries of oligonucleotides.

### CROSS REFERENCE TO RELATED APPLICATIONS

The present application claims priority from U.S. Provisional Application No. 60/837,108, filed Aug. 10, 2006 and U.S. Provisional Application 60/736,785, filed Nov. 15, 2005. Applicants claim the benefits of priority under 35 U.S.C. § 119 as to the provisional applications, and the entire disclosures of each of these applications are incorporated herein by reference in their entireties.

### FIELD OF THE INVENTION

The present invention relates to a method of reducing the complexity of a nucleic acid sample in a reproducible manner by enriching for specific nucleic acid target sequences in the population. Specifically it relates to a method to enrich for specific target sequences using libraries of oligonucleotides such as micro-arrays, for example, for use in sequencing and particularly sequencing by synthesis.

### BACKGROUND TO THE INVENTION

The draft sequence of the human genome was published in 2001 by the Human Genome Consortium (Nature Vol 409; issue 6822) and Celera genomics (Science, Vol 291, Issue 5507, 1304-1351), thus marking the beginning of the genetics chapter for society. Capitalizing on this investment and realizing the potential of the Human Genome Project requires a better understanding of genetic variation and its effect in disease.

It has been estimated that any two copies of the human genome differ from one another by as little as 0.1%, in other words a total of three million variants, or one variant every 1000 bases, over a total of three billion that make up the human genome. The consensus sequence of the human genome was based on information from just 12 genomes (six individuals; each person has two genomes, one from each parent), yet today there are six billion individuals worldwide (Bennet, S., Current Drug Discovery, February 2004, p15-19).

Since such variation affects disease susceptibility and responses to drugs it is essential to identify the genetic factors which contribute to biological variation. DNA sequencing is a fundamental tool enabling the screening of genes for such genetic mutations associated with disease. High throughput, high accuracy sequencing methods are therefore required to screen the complete genome sequence of an animal in order to identify unique nucleic acid sequences which may indicate the presence of physiological or pathological conditions.

DNA sequencing of large and complex genomes is currently limited by cost. With a significant proportion of human genomic DNA comprising repetitive sequence, reducing the complexity of the sample reduces the amount of sequencing required. Furthermore, with prior genetic information, it is possible to correlate a phenotype, such as a predisposition to a disease, with the genetic variation of one or more regions of the genome, and what is desired is the application and advantages of high throughput sequencing methods specifically to these regions of interest among many individuals. Such studies are currently not feasible due to cost. In addition, in certain circumstances, it is desirable to generate a ‘genome-wide’ analysis of a particular genomic feature, such as exons, to correlate genetic diversity in the protein-coding regions across many individuals.

Consequently development of strategies that focus on targeted sequencing of gene rich regions provide an alternative approach to whole genome sequencing.

Weisburg et al, U.S. Pat. No. 6,534,273, describe a method for capturing a target polynucleotide present in a sample onto a solid support with an attached immobilised probe by using a capture probe and two different hybridisation conditions that control the order of hybridisation, where the first hybridisation condition allows hybridisation of the capture probe to the target polynucleotide, and the second hybridisation condition allows hybridisation of the capture probe to the immobilised probe. The method further includes amplifying the captured target polynucleotide by hybridising at least one primer oligonucleotide to the target polynucleotide and using nucleic acid amplification that initiates from the primer oligonucleotide. The method utilises two separate probes for use in diagnostic assays, for example in testing for the presence of bacteria in a biological sample.

Collins et al, U.S. Pat. No. 5,750,338, describe a method of assay for target polynucleotides which includes the steps of isolating target polynucleotides from extraneous non-target polynucleotides, debris, and impurities and amplifying the target polynucleotide. The method provides for detection of nucleic acid targets in clinical samples.

Urdea, U.S. Pat. No. 5,200,314, describes an analyte polynucleotide strand having an analyte sequence which is detected within a sample containing polynucleotides by contacting the analyte polynucleotide with a capture probe under hybridization conditions, where the capture probe has a first binding partner specific for a solid-phase second binding partner.

The resulting duplex is then immobilized by specific binding between the binding partners, and non-bound polynucleotides are separated from the bound species. The analyte polynucleotide is optionally displaced from the solid phase, then amplified by PCR. The PCR primers each have a polynucleotide region capable of hybridizing to a region of the analyte polynucleotide, and at least one of the primers further has an additional binding partner capable of binding a solid-phase binding partner. The amplified product is then separated from the reaction mixture by specific binding between the binding partners, and the amplified product is detected.

Nisson et al, U.S. Pat. No. 6,268,133, discloses the use of amino acid denaturants for denaturing or separating double stranded nucleic acid molecules and more specifically, provides a method for the rapid isolation and recovery of a desired target DNA or RNA molecules from a mixture or library containing such molecules. The method involves the use of haptenylated probes and amino acid denaturants to select the desired molecules and eliminate the undesired library members from a sample. Their invention also provides a method in which larger or full-length nucleic acid molecules can be isolated from the subpopulation of desired molecules.

WO01/46470 in the name of Karolinska Innovations relates to a method for enrichment of specific nucleic acid segments, such as a DNA, e.g. single nucleotide polymorphisms (SNPs), sequences that have been deleted, sequences that are identical between two complex genomes, etc. The disclosed method includes steps for providing a first sample A and a second sample B derived from different sources and digestion of both said samples; amplification of sample A with a suitable primer and dNTPs comprising one unconventional base and amplification of sample B with a labelled primer and all the conventional dNTPs, followed by combination of samples A and B; denaturation and hybridization; treatment with a nuclease specific for said unconventional base, such as uracil-DNA glycosylase (UDG), and isolation of the specific segment originally present in sample B by use of the primer label. Their invention further relates to a kit which comprises components suitable for working the above described method.

WO02/06528 in the name of Somalogic, Inc. relate to a method and apparatus for the automated generation of nucleic acid ligands. The disclosure includes a method and device for performing automated SELEX. The steps of the SELEX process are performed at one or more work stations on a work surface by a robotic manipulator controlled by a computer. The document also includes methods and reagents to obviate the need for size-fractionation of amplified candidate nucleic acids before beginning the next round of the SELEX process. SELEX or Systematic Evolution of Ligands by EXponential enrichment, is a procedure in which an initial pool of randomized polynucleotides (RNA or DNA, single stranded) is created, containing on the order of 10ˆ15 molecules of a fixed length. The pool is then screened for some desired characteristic for example, binding affinity for ATP. The molecules that are selected in this way are used as “parents” in the synthesis (with mutation) of a new pool of molecules, and the process repeats with more rounds of selection and amplification. The result of SELEX is a set of highly functional molecules of DNA or RNA that perform their selected function.

U.S. Pat. No. 6,013,440, Lipshutz et al, relates generally to matrices for conducting nucleic acid affinity chromatography. Specifically the invention relates to methods of preparing affinity chromatography matrices that bind a plurality of different pre-selected nucleic acids.

Su et al, U.S. Pat. No. 6,632,611, disclose methods and kits for amplifying a target sequence from within a nucleic acid population. The invention provides selection probes which are complementary to at least a portion of said target sequence and mechanisms for adding a probe sequence to the 3′ end of a target sequence that is hybridized to a selection probe.

The added 3′ probe sequence and a probe sequence added at the 5′ end of the target by adaptor ligation allow for selective amplification of the target sequence.

Morgan et al (1992) disclose methods to direct cDNA selection allowing rapid and reproducible isolation of low abundance cDNA's encoded by large genomic clones.

Gill et al (2002) disclose a DNA microarray method for genome-wide monitoring of competitively grown transformants to identify genes whose overexpression confers a specific cellular phenotype.

These documents disclose methods of enriching a nucleic acid sample, usually by amplification, for the detection of specific target sequences. None of them provide a rapid, cost-effective method for reducing the complexity of a nucleic acid sample suitable for sequencing and particularly sequencing by synthesis. Furthermore, no methods have been described that utilise sample enrichment with high throughput sequencing methodology, such as, for example, reversible terminator chemistry described herein.

### SUMMARY OF THE INVENTION

In a first aspect of the invention there is provided a method of enriching a complex nucleic acid sample for a population of target sequences for use in subsequent sequencing wherein each of said target sequences relates to a set of capture probes.

Said method comprises:

(a) fragmenting a first population of nucleic acid sequences;
(b) combining said first population of nucleic acid sequences with a set of probe sequences under conditions allowing for hybridisation of the probe sequences and said first population of nucleic acid sequences to form probe-target complexes; and
(c) purifying the probe-target complexes to discard the un-hybridised nucleic acid target sequences;
(d) sequencing the remaining probe selected population of target sequences.

In one embodiment of the invention, the method further comprises a ligation step wherein adaptors are ligated to the fragmented first population of nucleic acid sequences, either prior to or subsequent to the enrichment step.

In a further embodiment of the invention the method further comprises an amplification step whereby the fragmented first population of nucleic acid sequences are amplified using, for example, PCR. Preferably said amplification step is performed following ligation of adaptors to the fragmented first population of nucleic acid sequences. Yet more preferably said amplification step is performed on the first population of nucleic acid sequences as a whole and in contrast to the methods of the prior art is not intended to amplify only a subset of said first population.

In another embodiment of the invention the target sequences can be removed from the probe-target complex prior to sequencing, for example by elution. Removal by denaturation of the selected targets from the immobilised capture probes will generally give a solution of single stranded targets.

In another embodiment of the invention the method further comprises the step of ligating adaptors to the enriched target sequences after separation of said target sequences from the probe target complexes.

In another embodiment of the invention the target sequences remain bound to the probe(s) and are sequenced directly on the array using, for example, sequencing by synthesis (SBS).

In yet another embodiment of the invention the target sequences are removed from the array and are further amplified and/or immobilised to produce clustered arrays, or sequenced directly as single molecules.

In still yet another embodiment of the invention, enrichment of a first population of nucleic acid sequences and subsequent sequencing of target sequences takes place on a single surface i.e. a single array or ‘chip’.

### BRIEF DESCRIPTION OF THE DRAWINGS

FIG. 1a illustrates a simplified and schematised embodiment of the use of a microarray to enrich a fragmented complex nucleic acid sample for a population of target sequences.

FIG. 1b illustrates a simplified and schematised embodiment of the use of a microarray to enrich a fragmented complex nucleic acid sample for a population of target sequences wherein adaptors are ligated to the fragmented genomic DNA prior to enrichment.

FIGS. 2a and 2b illustrate a simplified and schematised embodiment of the use of a microarray for ‘one-chip’ enrichment and sequencing. Genomic DNA is fragmented, adaptors are ligated to the ends of the fragments which are then amplified. The capture probes hybridise to the target sequences which are then extended to produce a complimentary sequence bound to the capture probe. The capture probe can then be bound to the surface of an array, target sequence is removed and the complimentary sequence is sequenced.

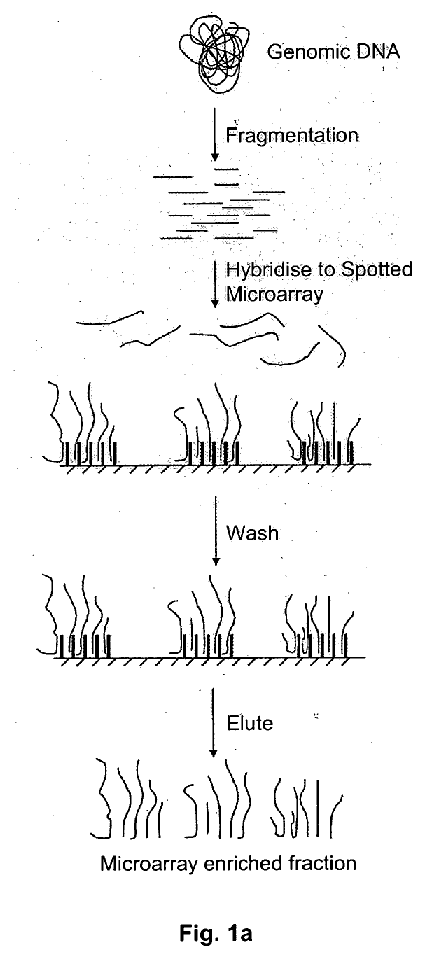

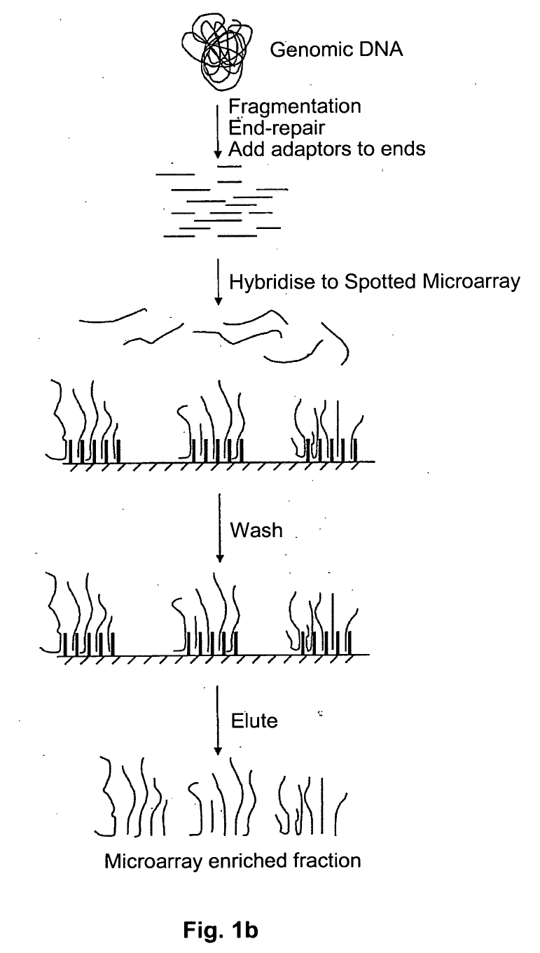

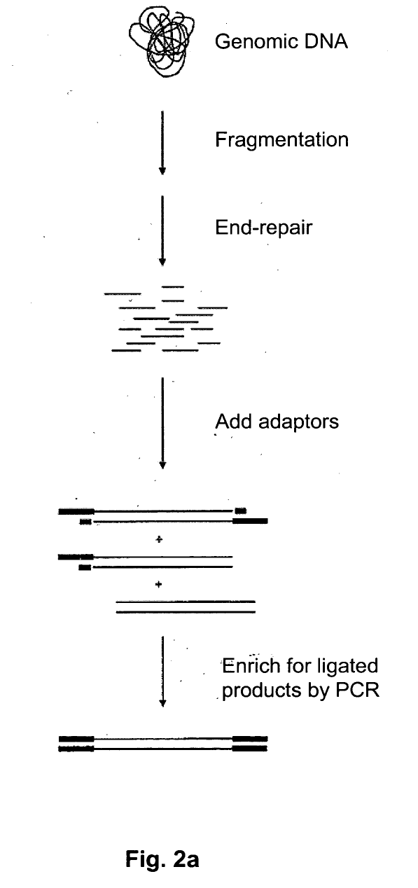

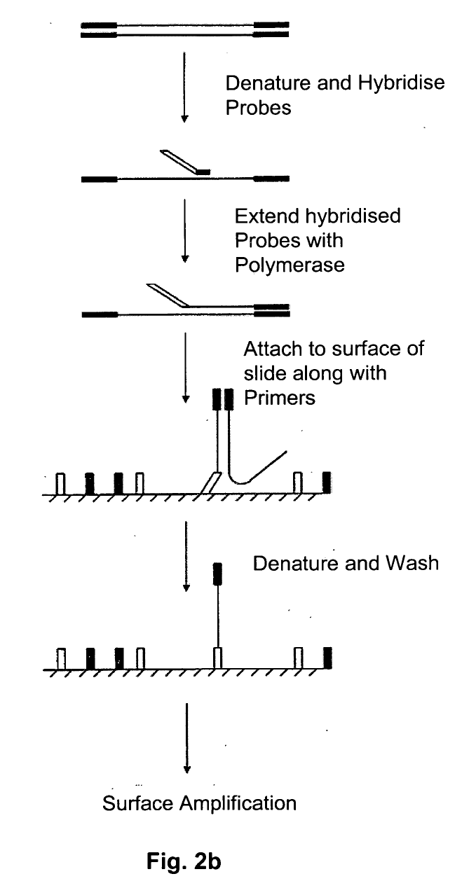

### DETAILED DESCRIPTION OF THE INVENTION

It is an object of the present invention to provide a method for enrichment of a complex nucleic acid sample with capture probes, relating for example to genetic features of interest. More specifically the invention relates to the use of a predetermined panel of oligonucleotides, such as an array, designed to enrich said complex nucleic acid sample for use in sequencing and more particularly sequencing by synthesis.

Micro-arrays have been used primarily for gene expression analyses, although the strategy of using an ordered array of bio-molecules on such an array has also been extended to mutation detection, polymorphism analysis, mapping and evolutionary studies.

To date however there has been no disclosure on the use of such arrays to enrich a complex nucleic acid sample with specific nucleic acid sequences relating to genetic features of interest for use in sequencing by synthesis.

The use of capture probes allows the researcher to positively select for regions of the genome which are of interest whilst concomitantly negatively selecting for the remainder of the genome. Such an approach has the advantage, for example, that highly repetitive DNA sequences which comprise 40% of genomic DNA can be removed quickly and efficiently from a complex population. As a direct result the complexity of sequence is reduced thus increasing throughput of subsequent sequencing. However, further enrichment for a target region or feature, such as exons, would further reduce the complexity of the sample. Usually, and in contrast to expression analysis studies for example, the nucleic acid sample applied to the array will not first be fluorescently labelled.

Since the method may be performed using micro-arrays there is the added benefit that the volume of input sample required is significantly reduced over methods of the prior art. The ability to use smaller quantities of input sample is a significant advantage over techniques in the prior art which often require complex strategies to increase the amount of nucleic acid by amplification. A further advantage is that the cost of carrying out both the enrichment and subsequent sequencing is also significantly reduced since less sequence data needs to be generated to produce meaningful results.

The term ‘enrichment’ refers to the process of increasing the relative abundance of particular nucleic acid sequences in a sample relative to the level of nucleic acid sequences as a whole initially present in said sample before treatment. Thus the enrichment step provides a percentage or fractional increase rather than directly increasing for example, the copy number of the nucleic acid sequences of interest as amplification methods, such as PCR, would. The methods as described herein may be used to remove DNA strands that it is not desired to sequence, rather than to specifically amplify only the sequences of interest. At the level of the whole genome, removing 50% of the DNA sample gives a two fold reduction in the cost and time of sequencing the remaining regions of biological interest from the whole genome. The methods as described herein can also be used to select large regions of a genome (eg megabases) for resequencing of multiple individuals, or can select out all the exons in a genomic sample. The synthesis of one array, or pool of oligonucleotides, can be used to process multiple samples of interest, and thus the costs of the oligonucleotide synthesis can be amortised over many individual samples.

The complex nucleic acid sample or input sample is an initial sample of nucleotide sequences prior to enrichment, such as genomic DNA. As non-limiting examples, such a sample may consist of genomic DNA, CDNA, RNA, PCR products, pools or subsets thereof.

In a first embodiment there is provided a method of enriching a complex nucleic acid sample for a population of target sequences for use in subsequent sequencing wherein each of said target sequences relates to a set of capture probes.

Said method comprises:
(a) fragmenting a first population of nucleic acid sequences;
(b) combining said first population of nucleic acid sequences with a set of probe sequences under conditions allowing for hybridisation of the probe sequences and said first population of nucleic acid sequences to form probe-target complexes; and
(c) purifying the probe-target complexes to discard the un-hybridised nucleic acid target sequences;
(d) sequencing the remaining probe selected population of target sequences

Preferably said fragmented nucleic acid population comprises sequence fragments which are less than about 1000 base pairs in length, more preferably such sequences are in the range 100-1000 base pairs in length. Still more preferably such sequences are in the range of from 450-750 base pairs in length. It would be apparent to the skilled artisan that the following non-limiting fragmentation methods may be used: restriction endonucleases, other suitable enzymes, mechanical forms of fragmentation, such as nebulisation or sonication, or non-enzymatic chemical fragmentation.

In one embodiment, adaptors may be ligated to the fragmented first population of nucleic acid sequences, either prior to or subsequent to the enrichment step.

In a further embodiment the fragmented first population of nucleic acid sequences may be subjected to an amplification step using, for example, PCR. Preferably said amplification step is performed following ligation of adaptors to the fragmented first population of nucleic acid sequences. Yet more preferably said amplification step is performed on the first population of nucleic acid sequences as a whole and in contrast to the methods of the prior art is not intended to amplify only a subset of said first population.

The capture probes are preferably nucleic acids, such as oligonucleotides, capable of binding to a target nucleic acid sequence through one or more types of chemical bonds, usually through complementary base pairing, usually through hydrogen bond formation. Such probes may include natural or modified bases and may be RNA or DNA. In addition the bases in probes may be joined by a linkage other than a phosphodiester bond so long as it does not interfere with hybridisation. Thus probes may also be peptide nucleic acids (PNA) in which the constituent bases are joined by peptide bonds rather than phosphodiester linkages.

Capture probes are reference populations of nucleic acid sequences. These have been selected such that said probes relate to, by way of non-limiting examples, a set of genes of interest, all of the exons of a genome, particular genetic regions of interest, disease or physiological states and the like. For example it can be envisaged that such reference populations will include commercially available populations available as micro-arrays or ‘chips’ more commonly used in expression profiling such as the Affymetrix® Exon Gene-Chip®. The capture probes can also be synthesised as oligonucleotides in solution, and can be used either in solution or immobilised on beads. The beads could contain multiple copies of individual sequences, such that each beads contains a single, different sequence, or can just contain the whole pool of oligonucleotides immobilised on each bead such that each bead is the same mixture of sequences.

Capture probes may also be prepared from a sample of DNA from any source, for example bacterial artificial chromosomes (BACs), PCR fragments, whole chromosomes or cDNA libraries. Use of a suitably available nucleic acid sample that can be fragmented and enriched as described means that the same region can be re-sequenced from multiple individuals without the need for chemical synthesis of specific capture probes across that region.

Any available nucleic acid can be fragmented and undergo a ligation with an adaptor sequence to establish common known ends on each fragment. Such fragment libraries can be amplified using primers complementary to the known ends and modified with groups amenable to surface attachment, such as, for example, biotin. The fragment pools, once made single stranded, are attached to a suitably functionalised surface, such as, for example, streptavidin beads. If the bead pool is exposed to a single stranded target DNA sample, then the fragments of the target DNA sample complementary to the single stranded fragments immobilised on the beads will bind, and the non-complementary sequences will remain unbound in solution and can be easily separated from the immobilised fragments.

Thus removal of sequences that were not complementary to those fragments of the capture pool enriches the remaining, immobilised target DNA.

The hybridisation step may be performed either on the solid surface, such as on beads, to which the single stranded capture probes have been bound, or in solution. If the hybridisation is performed in solution, subsequent addition of beads results in binding of all the capture probes, either as duplexes with the target sample, or as single strands. The remainder of the target DNA which has not formed duplexes with one of the capture probes will not be able to bind to the beads. Unbound target sample can be removed from the beads by washing, for example, and the duplex sample can be treated to elute the hybridised target into solution.

The enriched sample may be eluted from the beads and can be attached to a surface and used for sequencing, either as arrays of single molecules, or amplified to form clustered arrays of clonal single molecules, for example as described in WO9844151. In an alternative embodiment the enriched sample may be amplified whilst still attached to the beads by, for example, emulsion phase PCR, or may be eluted from the beads and amplified in solution prior to surface attachment.

The terms ‘target’ or ‘target sequence’ refer to nucleic acid sequences of interest that is, those which hybridise to the capture probes. Thus the term includes those larger nucleic acid sequences, a sub-sequence of which binds to the probe and/or to the overall bound sequence. Since the target sequences are for use in sequencing methods, said target sequences do not need to have been previously defined to any extent, other than the bases complementary to the capture probes.

Capture probes hybridise to target sequences in the complex nucleic acid sample. It will be apparent to one skilled in the art that prior to hybridisation said complex nucleic acid sample will preferably comprise single stranded nucleic acid sequences. This can be achieved by a number of well known methods in the art such as, for example using heat to denature or separate complementary strands of double stranded nucleic acids, which on cooling can hybridise to the capture probes. It is also conceivable that said complex nucleic acid sample could comprise double stranded polynucleotides with a single stranded overhang (‘sticky ends’) which may hybridise to said capture probes.

To provide enrichment, the capture probes are preferably immobilised onto a support, either before or after hybridisation, such that sequences that do not hybridise to said capture probes can be removed for example, by washing.

In one embodiment the target sequences can be removed from the probe-target complex prior to sequencing for example by elution. Removal by denaturation of the selected targets from the immobilised capture probes will generally give a solution of single stranded targets.

In a further embodiment adaptors may be ligated to the enriched target sequences after removal of said target sequences from the probe target complexes. The target sequences may also be further fragmented after elution from the support used for enrichment. For example, it may be advantageous to initially fragment the target sample to an average size of 10 kB, and thereby require fewer probe sequences to select out a specific megabase region. A 10 kB region can be selected, but not easily amplified, and therefore further fragmentation, to an average of a few hundred bases may be used after the enrichment step. If a second fragmentation step is used, then the universal adaptors will need to be ligated onto the enriched target sequences after the removal from the support and after the further fragmentation step.

The solid support may be any of the conventional supports used in arrays or ‘DNA chips’, beads, including magnetic beads or polystyrene latex microspheres, arrays of beads, or substrates such as membranes, slides and wafers made from cellulose, nitrocellulose, glass, plastics, silicon and the like.

Preferably the solid support is a flat planar surface or an array of beads. Still more preferably said solid support is an array and most preferably said array is a ‘high density array’ such as a micro-array.

Arrays are collections of biomolecular probes such as nucleic acids which are immobilised onto a solid support; as non-limiting examples, the biomolecular probes could be oligonucleotides of varying length (preferably 25 to 60mers), PCR products representing a cDNA clone library or BAC clones such as those used in comparative genome hybridisation. Multi-polynucleotide arrays or clustered arrays are ‘high density arrays’ of nucleic acid molecules which may be produced using techniques generally known in the art. By way of example, WO98/44151 and WO00/18957 both describe methods of nucleic acid amplification which allow amplification products to be immobilised on a solid support in order to form arrays comprised of clusters or ‘colonies’ of immobilised nucleic acid molecules. An array of amplified molecules from a previously enriched, or otherwise obtained target may be used to select the same target regions from a new sample. The enriched DNA can be sequenced directly on the array, or removed from the array for subsequent sequencing by any desired sequencing process.

Preferably said array contains greater than 100 probes. More preferably said array contains greater than 1000 probes, still more preferably said array contains greater than 10,000 probes. Still yet more preferably said array contains greater than 100,000 probes.

Immobilisation of the probes may be by specific covalent or non-covalent interactions. If the molecule is a polynucleotide, immobilisation will preferably be at either the 5′ or 3′ position so that the polynucleotide is attached to the solid support at one end only. However, the polynucleotide may be attached to the solid support at any position along its length, the attachment acting to tether the polynucleotide to the solid support. The immobilised polynucleotide is then able to undergo interactions with other molecules or cognates at positions distant from the solid support. Typically the interaction will be such that it is possible to remove any molecules bound to the solid support through non-specific interactions, e.g. by washing.

In one embodiment the target sequences remain bound to the probe and can be sequenced directly on the array using, for example, sequencing by synthesis (SBS). The can either be on a chemically synthesised array, or as a lawn of primers deposited such that a single molecule array of selected templates can be formed. Single molecule arrays and their use in sequencing is described in WO0006770.

In another embodiment the target sequences are removed from the array and may be optionally amplified in solution prior to immobilisation. The target sequences, and their complementary copies can be immobilised on a solid support. The immobilised arrays can be further amplified to produce clustered arrays, or sequenced directly as single molecules.

Any suitable method for of sequencing may be used to determine a sequence read of the immobilised enriched targets. Suitable methods of sequencing include the use of sequencing by addition of nucleotide bases, for example sequencing by synthesis (SBS) using nucleoside triphosphates (as described in WO04018497) and DNA polymerases, or using oligonucleotide cassettes and ligases; as described in U.S. Pat. No. 6,306,597 or Science, 309:5741, 1728-1732 (2005). The enriched targets may also be sequenced by pyrosequencing (Nature. 437:376-380 (2005)), or by MPSS where the strands are degraded rather than extended (Nat Biotechnol. 6:630-6344 (2000)).

In “sequencing by synthesis” or SBS a new polynucleotide strand based-paired to a template strand is built up in the 5′ to 3′ direction by successive incorporation of individual nucleotides complementary to the template strand. In one embodiment of SBS the substrate nucleoside triphosphates used in the sequencing reaction are each labelled on the base with different labels permitting determination of the identity of the incorporated nucleotide as successive nucleotides are added. The labelled nucleoside triphosphates also have a 3′ blocking group which prevents further incorporation of complementary bases by the polymerase. The label of the incorporated base can then be determined and the blocking group removed to allow further polymerisation to occur.

There are known in the art methods of nucleic acid sequencing based on successive cycles of incorporation of fluorescently labelled nucleic acid analogues. In such “sequencing by synthesis” or “cycle sequencing” methods the identity of the added base is determined after each nucleotide addition by detecting the fluorescent label. In particular, U.S. Pat. No. 5,302,509 describes a method for sequencing a polynucleotide template which involves performing multiple extension reactions using a DNA polymerase to successively incorporate labelled polynucleotides complementary to a template strand.

The present inventors have developed methods of sequencing multiple nucleic acid molecules in parallel based on the use of arrays, wherein multiple template molecules immobilised on the array are sequenced in parallel. Such arrays may be single molecule arrays or clustered arrays. The nucleotide (s) incorporated into the strand of nucleic acid complementary to the template nucleic are each fluorescently labelled. The inclusion of a fluorescent label facilitates detection/identification of the base present in the incorporated nucleotide(s). Appropriate fluorophores are well known in the art.

The labels may be the same for each type of nucleotide, or each nucleotide type may carry a different label. This facilitates the identification of incorporation of a particular nucleotide. Thus, for example modified adenine, guanine, cytosine and thymine would all have attached a different fluorophore to allow them to be discriminated from one another readily. When sequencing on arrays, a mixture of labelled and unlabelled nucleotides may be used. Detectable labels such as fluorophores can be linked to nucleotides via the base using a suitable linker. The linker may be acid labile, photolabile or contain a disulfide linkage. Preferred labels and linkages include those disclosed in W003/048387. Other linkages, in particular phosphine-cleavable azide-containing linkers, may be employed in the invention as described in greater detail in W02004/018493. The contents of WO 03/048387 and WO 2004/018493 are incorporated herein in their entirety by reference.

The nucleotides described in W02004/018493 comprise a purine or pyrimidine base and a ribose or deoxyribose sugar moiety which has a removable blocking group covalently attached thereto, preferably at the 3′O position. 3′ blocking groups are also described in W02004/018497, the contents of which are also incorporated herein in its entirety by reference. Use of such 3′-blocked nucleotides permits controlled incorporation of nucleotides in a stepwise manner, since the presence of a blocking group at the 3′-OH position prevents incorporation of additional nucleotides. The detectable label may, if desirable, be incorporated into the blocking groups as is disclosed in W02004/018497.

In further embodiments of SBS or cycle sequencing wherein the substrate nucleoside triphosphates used in the sequencing reaction are each labelled on the base with the same label and/or wherein the labelled nucleoside triphosphates do not have a 3′ blocking group to prevent further incorporation of complementary bases by the polymerase it will be apparent to the skilled person that in these cases the nucleotides can be supplied individually and serially and incorporation of a base can then be determined before applying the next nucleotide.

Methods for detecting fluorescently labeled nucleotides generally require use of incident light (e.g. laser light) of a wavelength specific for the fluorescent label, or the use of other suitable sources of illumination, to excite the fluorophore. Fluorescent light emitted from the fluorophore may then be detected at the appropriate wavelength using a suitable detection system such as for example a Charge-Coupled-Device (CCD) camera, which can optionally be coupled to a magnifying device, a fluorescent imager or a confocal microscope. If sequencing is carried out on an array, detection of an incorporated base may be carried out by using a confocal scanning microscope to scan the surface of the array with a laser, to image fluorescent labels attached to the incorporated nucleotide(s). Alternatively, a sensitive 2-D detector, such as a charge-coupled detector (CCD), can be used to visualise the signals generated. This technique is particularly useful with single molecule arrays.

Other techniques such as scanning near-field optical microscopy (SNOM) are available and may be used when imaging dense arrays. For a description of scanning near-field optical microscopy, see Moyer et al., Laser Focus World 29:10, 1993. An additional technique that may be used is surface-specific total internal reflection fluorescence microscopy (TIRFM); see, for example, Vale et al., Nature, (1996) 380:451-453.

Suitable apparatus used for imaging polynucleotide arrays are known in the art and the technical set-up will be apparent to the skilled person. Detection buffers containing antioxidants, such as sodium ascorbate, show a clear improvement (over corresponding buffers absent such antioxidants) at preventing light-induced chemical artefacts in cycles of sequencing-by-synthesis based on detection of fluorescently labeled nucleotide analogues, as described in WO06064199. The inclusion of antioxidants prevents/reduces light-induced chemical reactions from damaging the integrity of the nucleic acid template and allows accurate determination of the identity of the incorporated base over at least 2, preferably at least 10 and more preferably at least 16 cycles of nucleotide incorporation.

Preferably from 10 to 50 and more preferably from 16 to 30 nucleotides are successively incorporated, and identified, in the sequencing reaction. The ability to accurately sequence 10 or more, and preferably 16 or more, consecutive nucleotides in a sequencing reaction is a significant advantage in applications such as genome re-alignment. In the context of this invention the terms “sequencing reaction”, “sequencing methodology” or “method of sequencing” generally refer to any polynucleotide “sequencing-by-synthesis” reaction which involves sequential addition of one or more nucleotides or oligonucleotides to a growing polynucleotide chain in the 5′ to 3′ direction using a polymerase or ligase in order to form an extended polynucleotide chain complementary to the template nucleic acid to be sequenced.

The identity of the base present in one or more of the added (oligo)nucleotide(s) is determined in a detection or “imaging” step. The identity of the added base is preferably determined after each nucleotide incorporation step. The sequence of the template may then be inferred using conventional Watson-Crick base-pairing rules.

The nucleic acid template to be sequenced in a sequencing reaction may be any polynucleotide that it is desired to sequence. The nucleic acid template for a sequencing reaction will typically comprise a double stranded region having a free 3′ hydroxyl group which serves as a primer or initiation point for the addition of further nucleotides in the sequencing reaction. The region of the template to be sequenced will overhang this free 3′ hydroxyl group on the complementary strand. The primer bearing the free 3′ hydroxyl group may be added as a separate component (e.g. a short oligonucleotide) which hybridises to a region of the template to be sequenced. Alternatively, the primer and the template strand to be sequenced may each form part of a partially self-complementary nucleic acid strand capable of forming an intramolecular duplex, such as for example a hairpin loop structure. Nucleotides are added successively to the free 3′ hydroxyl group, resulting in synthesis of a polynucleotide chain in the 5′ to 3′ direction. After each nucleotide addition the nature of the base which has been added may be determined, thus providing sequence information for the nucleic acid template.

The term “incorporation” of a nucleotide into a nucleic acid strand (or polynucleotide) refers to joining of the nucleotide to the free 3′ hydroxyl group of the nucleic acid strand via formation of a phosphodiester linkage with the 5′ phosphate group of the nucleotide. The nucleic acid template to be sequenced may be DNA or RNA, or even a hybrid molecule comprised of deoxynucleotides and ribonucleotides. The nucleic acid may comprise naturally occurring and/or non-naturally occurring nucleotides and natural or non-natural backbone linkages.

Nucleic acid templates to be sequenced may be attached to a solid support via any suitable linkage method known in the art. Preferably linkage will be via covalent attachment. If the templates are “arrayed” on a solid support then the array may take any convenient form. Thus, the method of the invention is applicable to all types of “high density” arrays, including single-molecule arrays and clustered arrays.

The enrichment method of the invention may be carried out using essentially any type of array formed by immobilisation of nucleic acid molecules on a solid support, and more particularly any type of high-density array, including bead arrays. The sequencing aspect of the invention may be carried out using essentially any type of array formed by immobilisation of nucleic acid molecules on a solid support, and more particularly any type of high-density array, including single molecule, amplified single molecule (cluster) arrays, arrays of beads on which molecules have been amplified (for example in an emulsion PCR reaction), or arrays of beads on which amplified molecules have been hybridised.

In multi-polynucleotide or clustered arrays distinct regions on the array comprise multiple copies of single polynucleotide template molecules. Multi-polynucleotide or clustered arrays of nucleic acid molecules may be produced using techniques generally known in the art. By way of example, WO98/44151 and WO00/18957 both describe methods of nucleic acid amplification which allow amplification products to be immobilised on a solid support in order to form arrays comprised of clusters or “colonies” of immobilised nucleic acid molecules. The arrays are amplified such that both strands of a duplex are immobilised, but cleavage of one of the strands from the surface (for example using a chemical and/or a subsequent heat treatment to cleave and denature one of the amplification primers used to generate the copies of the immobilised single molecules), results in an array of single stranded templates suitable for sequencing.

The nucleic acid molecules present on the clustered arrays prepared according to these methods are suitable templates for sequencing using the method of the invention. Both WO98/44152 and WO00/18957, the contents of which are incorporated herein by reference describe methods of parallel sequencing of multiple templates located at distinct locations on a solid support, and in particular sequencing of “clustered” arrays. The single stranded arrays described above can be hybridised with a suitable sequencing primer, complementary to a region common to each of the amplified templates, to provide a free 3′-hydroxyl group suitable for sequencing against the unknown, variable region of the amplified templates.

Nevertheless, the method of the invention may also be used in the context of sequencing templates on single molecule arrays of nucleic acid templates. Single molecule arrays are generally formed by immobilisation of a single polynucleotide molecule at each discrete site that is detectable on the array. Single-molecule arrays comprised of nucleic acid molecules that are individually resolvable by optical means and the use of such arrays in sequencing are described, for example, in WO00/06770.

Single molecule arrays comprised of individually resolvable nucleic acid molecules including a hairpin loop structure are described in WO 01/57248. The method of the invention is suitable for sequencing template molecules on single molecule arrays prepared according to the disclosures of WO 00/06770 or WO 01/57248. The fluorescent moiety may be attached to a nucleic acid via any suitable covalent or non-covalent linkage. For example, the fluorescent moiety may be attached to an oligonucleotide primer or probe which is hybridised to a target nucleic acid molecule.

In a preferred embodiment, enrichment of a first population of nucleic acid sequences and subsequent sequencing of target sequences takes place on a single surface i.e. a single array or ‘chip’. More preferably the sequencing is by sequencing by synthesis.

Preferably a large number of target sequences are sequenced in parallel at the same time. More preferably greater than 100 target sequences are sequenced at a time. More preferably greater than 1000 target sequences are sequenced at one time, still more preferably greater than 10,000 target sequences are sequenced at one time. Still yet more preferably greater than 100,000 target sequences are sequenced at one time.

Experimental Overview

The following experimental details describe the complete exposition of one embodiment of the invention as described above.

### EXAMPLE 1

Four probes can be designed that hybridise to the 5′ end of one exon from each of the four following genes present in the human BAC BCX98J21: PPP1R10, ABCF1, PRR3, GNL1. Each probe is 60 bases in length, contains a 5′ biotin group and hybridises uniquely at its intended sequence in the BAC. The sequences of the probes are as follows:

Probe #1 (PPP1R10)
TCGGTTAAGGAAGCTGTCCAGGCCCTTGAGAAGTTCTTTGGGGTCTATGG
GACCCGAACC
Probe #2 (ABCF1)
GCCGTATCTGAGGAACAGCAGCCTGCACTCAAGGGCAAAAAGGGAAAGGA
AGAGAAGTCA
Probe #3 (PRR3)
CCGAAACGAAAGAAGCAGAATCATCACCAGCCACCGACACAGCAGCAGCC
CCCGCTGCCC
Probe #2 (GNL1)
CTCCCGTTTGTCCTGCAACTGCTTCTTCTTCTGCTTCACGCTGAATGGCT
TCTTCCTCGG

A solution is prepared containing a mixture of all four probes at a concentration of imicromolar each in 5×SSC buffer and is added to a tube containing 1 microgram of BAC DNA that has been previously fragmented to less than 1000 base pairs using a nebulizer (Invitrogen® #K7025-05) in a total volume of 50 ul. The solution is heated to 97.5° C. for 5 minutes, then cooled to room temperature to anneal the probes to their target sequences in the BAC fragments.

Wash 40 ul of streptavidin coated magnetic beads (Dynal® Biotech) twice with 100 ul of 2×B&W buffer (Dynal®), finally resuspending the beads in 50 ul of 2×B&W. Add to the solution of annealed DNA and incubate for 15 minutes at room temperature with gentle mixing on a roller mixer platform. Apply the tube of the DNA-bead mix to the magnetic holder (Dynal®) and incubate at room temperature for 2 minutes. Wash the beads three times with 1×B&W buffer (Dynal®), each time discarding the supernatant, resuspending the beads in fresh 1×B&W buffer and reapplying to the magnets. Finally, add 50 ul of 100 mM NaOH to the DNA-bead mix and incubate at room temperature for 5 minutes. Reapply to the magnetic holder, then recover the supernatant after a 2 minute incubation, discarding the beads. The DNA recovered in the 100 mM NaOH solution may be neutralised by the addition of a titrated quantity of HCl. Alternatively, the DNA-NaOH solution can be exchanged for 5×SSC buffer by using a MicroSpin® S300 HR column (AmershamPharmacia® #27-5130-01).

### EXAMPLE 2

A set (500,000) of probes can be designed that hybridise to unique positions among the 10 regions of the human genome selected by the HapMap ENCODE resequencing and genotyping project. Each probe approximately 60 bases in length and contains a 5′ phosphorothioate group.

A solution is prepared of a mixture of all probes at a total concentration of 10 micromolar in 100 mM potassium phosphate buffer pH7. The probe set is grafted onto the surface of an array chip by flowing the solution of probes over the functionalised array surface at 15 ul/min at 51° C. The chip is then washed by pumping consecutively across the surface of the array: 100 mM potassium phosphate buffer pH7, TE buffer (10 mM Tris pH8, 10 mM EDTA) and 5×SSC.

1 ug of total human DNA can be fragmented to less than 1000 base pairs using a nebulizer (Invitrogen #K7025-05). The DNA is diluted to 10 nM in 5×SSC and then pumped onto the surface of the array. The array is heated to 97.5° C. for 5 minutes, then cooled to room temperature to anneal the fragmented total human DNA to the probe sequences on the surface of the array. Non-hybridised DNA is then removed by washing the surface of the array consecutively with the following solutions: 5×SSC, 0.3×SSC, and 5×SSC. The DNA that has hybridised to the surface probes can be recovered by pumping TE (10 mM Tris pH8, 1 mM EDTA) onto the array and heating the array to 97.5° C. for 5 minutes. Immediately thereafter, the contents of the array are pumped into a collecting tube at 97.5° C. and cooled to 4° C.

### EXAMPLE 3

1 ug of total human DNA can be fragmented to less than 1000 base pairs using a nebulizer (Invitrogen #K7025-05). The DNA is diluted to 1 nM in 5×SSC and then pumped onto the surface of an Affymetrixl® Genechip® Exon Array spotted microarray. The array is heated to 97.5° C. for 5 minutes, then cooled to 45° C. The array is further incubated at 45° C. for 16 hours to anneal the fragmented total human DNA to the primer oligonucleotides on the surface of the array. Non-hybridised DNA is then removed by washing the surface of the array with 3 cycles of the following consecutive wash solutions: 6×SSPE/0.01% Tween-20, 100 mM MES/0.01% Tween-20. The DNA that has hybridised to the surface probes can be recovered by pumping TE (10 mM Tris pH8, 1 mM EDTA) onto the array and heating the array to 97.5° C. for 5 minutes. Immediately thereafter, the contents of the array are pumped into a collecting tube at 97.5° C. and cooled to 4° C.

### EXAMPLE 4

1 ug of total human DNA can be fragmented to less than 1000 base pairs using a nebulizer (Invitrogen #K7025-05). The DNA is diluted to 1 nM in 5×SSC and then pumped onto the surface of an Affymetrixl® Genechip® Exon Array spotted microarray. The array is heated to 97.5° C. for 5 minutes, then cooled to 45° C. The array is further incubated at 45° C. for 16 hours to anneal the fragmented total human DNA to the primer oligonucleotides on the surface of the array. Non-hybridised DNA is then removed by washing the surface of the array with 3 cycles of the following consecutive wash solutions: 6×SSPE/0.01% Tween-20, 100 mM MES/0.01% Tween-20. The chip is then subject to multiple cycles of SBS sequencing.

### EXAMPLE 5

The following experimental details describe the complete exposition of one embodiment of the invention. The DNA source used is purified Human cell line DNA supplied by the Coriell Cell Repositories, Camden, N.J. 08103 USA, catalog no. NA07055. The DNA is first prepared for the ligation reaction to a single adaptor by: fragmentation of the DNA by nebulisation, then polishing of the DNA ends to make them blunt-ended and phosphorylated. The ligation reaction is performed with the prepared fragmented DNA and an adaptor preformed by annealing ‘Oligo A’ and ‘Oligo B’ (sequences given below). Next, the product of the ligation reaction is subject to cycles of PCR with a single primer ‘Oligo C’ (sequence given below) to selectively amplify ligated product that contains adaptor at both ends of the fragments. The product of the PCR reaction is purified from unligated adaptor and primer ‘Oligo C’ by gel electrophoresis. These products are next denatured, then renatured in the presence of a set of probes. The set (500,000) of probes can be designed such that they hybridise to unique positions among the 10 regions of the human genome selected by the HapMap ENCODE resequencing and genotyping project. Each probe is approximately 80 bases in length and contains a common (universal) sequence ‘Sequence D’ at the 5′ end as well as a terminal 5′ phosphorothioate group. After the probes have hybridised, a polymerisation reaction is performed with klenow polymerase and dNTPs to extend the hybridised probes to the 5′ end of the DNA fragments forming a duplex. The duplexes are next coupled to the surface of a DNA chip in conjunction with two amplification primers whose sequences are identical to the 5′ end of ‘Oligo A’ and ‘Sequence D’. The DNA coupled to the chip is denatured and washed to remove hybridised DNA. The chip can then be subjected to cluster amplification and sequencing by SBS.

Nebulization

Materials:

| | |
|---|---|
| Human DNA (1 mg/ml) | Corriell NMA07055 |
| Buffer (glycerol 53.1 ml, water 42.1 ml, | |
| 1 M TrisHCl pH 7.5 3.7 ml, 0.5 M | |
| EDTA 1.1 ml) | |
| Nebulizer | Invitrogen ® (#K7025-05) |
| Qiagen ® columns | PCR purification kit (#28104) |

Mix: 25 μl (5 micrograms) of DNA
   725 μl Buffer
      Procedure:

Chill the DNA solution and fragment in a nebulizer on ice for 5 to 6 minutes under at least 32 psi of pressure. Recover the solution by centrifugation (volume usually somewhere between 400 and 600 μl), split into 3 aliquots and purify with a Qiagen® PCR-purification kit, but using only one column, and finally elute in 30 μl of EB (Qiagen®).

End-Repair

Materials:

| | |
|---|---|
| T4 DNA Polymerase | NEB #M0203S |
| 10 × NEB 2 buffer | NEB #M7002S |
| 100 × BSA | NEB #M9001S |
| dNTPs mix (10 mM each) | NEB #N0447S |
| E. coli DNA Pol I large fragment | |
| (Klenow, NEB #M0210S) | |
| T4 polynucleotide kinase | NEB #M0201S |
| T4 PNK buffer | NEB #M0201S |
| 100 mM ATP | |
| Qiagen ® columns | PCR purification kit (#28104) |

End repair mix assembled as follows:

| | | |
|---|---|---|
| DNA | 30 μl | |
| Water | 12 μl | |
| 10 × NEB2 | 5 μl | |
| 100 × BSA | 0.5 μl | |
| 10 mM dNTPs | 2 μl | |
| T4 DNA pol (3 U/μl) | 5 μl | |
| | 50 μl | total |

Incubate the reaction for 15 min at room temperature, then add 1 μl of E. coli DNA Pol I large fragment (Klenow) and incubate the reaction for a further 15 min at room temperature. Purify the DNA from enzymes, buffer, etc by loading the reaction mix on a Qiagen® column, finally eluting in 30 μl EB. The 5′ ends of the DNA are then phosphorylated using polynucleotide kinase as follows:

| | | |
|---|---|---|
| DNA | 30 μl | |
| Water | 9.5 μl | |
| 10 × PNK buffer | 5 μl | |
| 100 mM ATP | 0.5 μl | |
| T4 PNK (10 U/μl) | 5 μl | |
| | 50 μl | Total |

Incubate the reaction for 30 min at 37° C., then heat inactivate at 65° C. for 20 min. DNA is then purified from enzymes, buffer, etc by loading the reaction mix on a Qiagen® column, finally eluting in 30 μl EB. Three separate tubes are pooled to give 90 μl total.

Anneal Adapter

Materials:

Oligo: 5′ AAATGATACGGCGACCACCGAGATCTACACTCTTTCCCTA
CACGACGCTCTTCCGATC
Oligo B: 5′ GATCGGAAGAGCGTCGTGTAG
50 mM Tris/50 mN NaCl pH7
PCR machine

| | |
|---|---|
| 100 μM Oligo A | 20 μl |
| 100 μm Oligo B | 20 μl |
| Tris/NaCl | 10 μl |
| | 50 μl at 40 μM duplex in 10 mM Tris/10 mM NaCl |
| | pH 7.5 |

The adapter strands are annealed in a PCR machine programmed as follows:

Ramp at 0.5° C./sec to 97.5° C.

Hold at 97.5° C. for 150 sec

Then a step of 97.5° C. for 2 sec with a temperature drop of 0.1° C./cycle for 775 cycles

Ligation Reaction

| | |
|---|---|
| 15 uM adaptor | |
| End-repaired fragmented | |
| DNA genomic DNA | |
| Quick Ligase | NEB #M2200L |
| Quick Ligase 2 × buffer | NEB #M2200L |
| PCR machine | |
| Qiagen ® columns | x1 PCR purification kit (#28104) |

| | | |
|---|---|---|
| DNA | 10 μl | |
| 2 × buffer | 25 μl | |
| 15 uM adaptor | 10 μl | |
| Quick Ligase | 5 μl | |
| | ˜50 μl | total |

Incubate for 40 min @ RT

Clean up with a Qiagen® column. Elute in 30 μl EB.

Pass down a S300 column to get rid of excess adaptor.

PCR Amplification

Materials:

Ligated DNA
Oligo C: AATGATACGGCGACCACCGA
2x RedTaq ™ PCR mix
PCR machine
Qiagen ® MinElute columns
Qiagen ® (#28004)

The purified ligated DNA is diluted 25 fold, then a PCR reaction mix prepared as follows:

| | | |
|---|---|---|
| DNA | 1 μl | |
| 2 + Red Taq ™ mix | 25 μl | |
| 100 μM Oligo C | 0.5 μl | |
| Water | 23.5 μl | |
| | ˜50 μl | total |

Thermocycling is carried out in a PCR machine under the following conditions:

2 min @ 70° C.

2 min @ 94° C.

[45 sec@ 94° C., 45 sec @ 65° C., 2 min @ 70° C.] 16 cycles

5 min @ 70° C.

Hold @ 4° C.

PCR products are purified from enzymes, buffer, etc on a Qiagen® MinElute® column, eluting in 10 μl EB.

Gel Purification

Materials:

| | |
|---|---|
| Agarose | Biorad ® #161-3101 |
| 100 base pair ladder | NEB #N3231L |
| TAE | |
| Loading buffer (50 mM Tris pH8, | |
| 40 mM EDTA, 40% w/v sucrose) | |
| Ethidium bromide | |
| Gel trays and tank. Electrophoresis unit | |

The entire sample from the purified PCR amplification reaction is loaded into one lane of a 2% agarose gel containing ethidium bromide and run at 120V for 50 min. The gel is then viewed on a ‘White-light®’ box and fragments from above 300 bp to at least 750 bp excised and purified with a Qiagen® Gel purification kit, eluting in 30 μl EB. For large gel slices two MinElute® columns are used, eluting each in 15 μl EB and pool.

Annealing of the Probe Sets

The probes all consist of the following format:

5′ PS-Sequence D-Probe sequence

where ‘PS’ represents a 5′ phosphorothioate and ‘Sequence D’ is as follows:

CAAGCAGAAGACGGCATACGA

The gel-purified DNA is denatured by adding NaOH at a final concentration of 100 mM and incubating at room temperature for 5 minutes. This solution is neutralised by adding 100 microlitres of pre-warmed hybridisation solution (6×SSPE, 0.1% Tween 20) containing the probe set at a final concentration of 10 micromolar, and incubated at 37° C. for 1 hour. The DNA is then purified on a Qiagen® MinElute® column, with a final elution in 10 microlitres of EB.

Probe Extension

Materials:

| | |
|---|---|
| 1 mM dNTPs mix | |
| 10 × buffer (100 mM Tris-HCl, pH 7.9, | |
| 100 mM MgCl2, 10 mM DTT, | |
| 500 mM NaCl) | |
| Klenow fragment (3′->5′ exo-) | NEB #M0212S |

Mix

| | | | |
|---|---|---|---|
| Probe-hybridised DNA | 10 | ul | |
| 10× buffer | 5 | ul | |
| 1 mM dNTP mix | 5 | ul | |
| H2O | 29 | ul | |
| Klenow exo- (2 Units/ul) | 1 | ul | |
| | ˜50 | μl | total |

Incubate at 37° C. for 1 hr, then purify on a Qiagen® DNA purification column, with a final elution in 30 ul of EB buffer.

Covalent Attachment to Array Chip

The coupling of DNA to the surface of a chip and sequencing of the template molecules on arrays may be carried out according to the disclosures of WO 00/06770, WO 01/57248 or WO06/064199 or PCT/GB2006/002687 the contents of which are herein incorporated by reference.

1. A method of obtaining a population of target sequences for the purpose of sequencing, wherein each of said target sequences relates to a pre-determined nucleic acid sequence of interest comprising:
(a) fragmenting a first population of nucleic acid sequences;
(b) combining said first population of nucleic acid sequences with a set of probe sequences under conditions allowing for hybridisation of the probe sequences and said first population of nucleic acid sequences to form probe-target complexes; and
(c) purifying the probe-target complexes to discard the un-hybridised nucleic acid target sequences;
(d) sequencing the remaining probe selected population of target sequences.

2. A method of obtaining a population of target sequences for the purpose of sequencing, wherein each of said target sequences relates to a pre-determined nucleic acid sequence of interest comprising:
(a) fragmenting a first population of nucleic acid sequences;
(b) combining said first population of nucleic acid sequences with a set of probe sequences under conditions allowing for hybridisation of the probe sequences and said first population of nucleic acid sequences to form probe-target complexes; and
(c) purifying the probe-target complexes to discard the un-hybridised nucleic acid target sequences;
(d) removing the bound sequences of the said first population of nucleic acid sequences from the probe-target complexes to form said population of target sequences
(e) immobilising said population of target sequences
(f) sequencing said immobilised population of target sequences.

3. A method of obtaining a selected population of target sequences for the purpose of sequencing, wherein each of said target sequences relates to a pre-determined nucleic acid sequence of interest comprising,
(a) fragmenting a first population of nucleic acid sequences;
(b) combining said fragmented nucleic acid population with a set of probe sequences under conditions allowing for hybridisation of the probe sequences and target sequences to form probe-target complexes;
(c) purifying the probe-target complexes from un-hybridised nucleic acid sequences by washing to leave probe-target complexes;
(d) removing the selected targets from the purified probe-target complexes;
(e) amplifying the target sequences to produce multiple copies of the selected population of target sequences;
(f) sequencing the multiple copies of the selected population of target sequences.

4. The method of claims 1 to 3, further comprising the step of ligating adaptors after fragmentation of nucleic acid sequences.

5. The method of claim 2 or 3, further comprising the step of ligating adaptors to the target sequences after removing said sequences from the probe-target complexes.

6. The method of claim 4, further comprising amplifying the nucleic acid sequences after ligating adaptor sequences.

7. The method of claim 5, further comprising amplifying the nucleic acid sequences after ligating adaptor sequences.

8. The method of claims 1 to 3, wherein said pre-determined probe sequences are immobilised on a support prior to hybridisation with the fragmented nucleic acid population.

9. The method of claims 1 to 3, wherein said probe-target complexes are immobilised on a support subsequent to hybridisation of the pre-determined probe sequences with the target sequences.

10. The method of claim 8, wherein said support is a solid support.

11. The method of claim 9, wherein said support is a solid support.

12. The method of claim 8, wherein said support is an array.

13. The method of claim 12, wherein said array is a high density array.

14. The method of claim 10, wherein said solid support is magnetic particles.

15. The method of claim 11, wherein said solid support is magnetic particles.

16. The method of claim 10, wherein said solid support is beads.

17. The method of claim 11, wherein said solid support is beads.

18. The method of claims 1 to 3, wherein said sequencing step comprises incorporation of one or more labelled nucleotide bases each having a reversible terminator attached thereto, and a suitable polymerase, and imaging to determine the identity of each incorporated base.

19. A method of using an array for enrichment of a nucleic acid population, wherein said using increases the relative abundance of specific target sequences in said nucleic acid population.

20. The method of claim 19, wherein said array is a high density array.

21. The method of claim 19, wherein the enriched nucleic acid population is used for sequencing by synthesis.
